(19) United States
(12) Patent Application Publication
(10) Pub. No.: US 2019/0366131 A1
(43) Pub. Date: Dec. 5, 2019

(54) PERSONAL CLEANSING CLOTHS

(71) Applicant: Medline Industries, Inc., Northfield, IL (US)

(72) Inventors: Sohum Mansukhani, Chicago, IL (US); Martin Coffey, Buffalo Grove, IL (US); Sharbel Maalouf, Pleasant Prairie, WI (US)

(21) Appl. No.: 15/993,859

(22) Filed: May 31, 2018

(51) Int. Cl. A61Q 19/10; A61K 8/02; A47K 7/03

(52) U.S. Cl. CPC A61Q 19/10; A45D 37/00; A47K 7/03; A61K 8/0208

(57) ABSTRACT

Disclosed is a package that comprises a plurality of personal cleansing cloths each impregnated with an aqueous cleansing composition that includes a skin cooling agent, a fragrance, a skin conditioning agent, and one or more botanical extracts. The botanical extract may comprise one or more of coffee seed extract, Punica Granatum extract, Croton Lechleri extract, Panax extract, and Crataegus Monogyna extract. In use, a personal cleansing method may comprise removing a personal cleansing cloth from the package and self-cleansing using the personal cleansing cloth.

### TECHNICAL FIELD

The invention is in the field of personal cleansing.

### BACKGROUND

Many individuals prefer to maintain an active lifestyle and regularly participate in activities such as exercise, sports, hiking, camping, and other activities involving physical exertion. It is sometimes inconvenient to bathe fully after participation in such activities. This can be problematic for someone returning to professional or social activities immediately after exercising or engaging in other physical activities. For example, some people wish to engage in exercise programs during a scheduled break in the business workday, but are unwilling to do so because of insufficient time to bathe before returning to work.

It has now been found that plurality of personal cleansing cloths may be provided in a package. The cleansing cloths may comprise a durable cloth material having a basis weight of at least 90 gsm and may be impregnated with an aqueous cleansing composition. The cleansing composition may include a skin cooling agent, a fragrance, a skin conditioning agent, and a botanical extract, the botanical extract comprising one or more of coffee seed extract, Punica Granatum extract, Croton Lechleri extract, Panax extract, and Crataegus Monogyna extract. A cloth material having the specified basis weight and impregnated with such cleansing composition is sufficiently durable to allow for personal bodily cleansing, while the skin cooling agent imparts a cooling sensation to the user to help cool the user's skin after physical exertion. The botanical extracts and fragrance impart a pleasant scent and to some extent may impart a deodorizing or odor-masking property.

In use, a personal cleansing method comprises providing a package described above, removing a personal cleansing cloth from the package, and self-cleansing using the personal cleansing cloth. The method is particularly applicable after exercise, sporting activity, and the like where there is insufficient time to bathe fully before returning to professional or social activities. The personal cleansing method is also useful, for example, for self-cleansing during the workday when the user feels it necessary.

### BRIEF DESCRIPTION OF THE DRAWING

FIG. 1 illustrates an exemplary package of cleansing cloths.

### DETAILED DESCRIPTION

A plurality of cleansing cloths are provided in a package. The package of cleansing cloths may include any suitable number of cloths, ranging from 1-50, for example. In some embodiments, ten cleansing cloths may be packaged in an individual package for retail sale. Each cleansing cloth should be composed of a durable material that is sufficient to withstand rigorous self-cleansing. It has been found that a needle-punched 50% rayon/50% polyester cloth having a basis weight of 110 gsm is suitable for this purpose. The basis weight of the material may range from as low as about 90 gsm to about 145 gsm, and the percentages of rayon and polyester may vary. Other suitable materials may include other conventional cloth products. Besides rayon/polyester substrates, other cloth substrates may include, for example, nonwoven fabric, paper, tissue, sponge or laminate of foam and fabric, including, polyethylene, polypropylene, polyester, viscose rayon, polylactic acid and nylon and any blends/combinations thereof. Non-limiting examples of synthetic fibers useful as components of the cloth include acetate fibers, acrylic fibers, cellulose ester fibers, modacrylic fibers, polyamide fibers, polyester fibers, polyolefin fibers, polyvinyl alcohol fibers, rayon fibers, acrylonitrile-based fibers, cellulose ester fibers polyamides, polyesters, and polyethylene terephthalate fibers.

The cloth is impregnated with an aqueous cleansing composition that includes at least a skin cooling agent, a botanical abstract comprising one or more of coffee seed extract, Punica Granatum extract, Croton Lechleri extract, Panax extract, and Crataegus Monogyna extract, a fragrance and a skin conditioning agent. The skin cooling agent may be, for example, menthol or menthyl lactate, and may be present in any amount effective to impart a cooling sensation to the skin. For example, the skin cooling agent may be present in an amount ranging from 0.05-0.1% by weight.

The skin conditioning agent may include a humectant, occlusive agent, or emollient, and multiple skin conditioning agents may be provided. The humectant may include ingredients such as propylene glycol, glycerin, urea, and pyrrolidone carboxylic acid (PCA). The humectant may be present in any amount suitable to attract water from the dermis and helping to keep that water bound in the Stratum corneum. For example, the humectant may be present in an amount ranging from 0.5-5% by weight, or 1-2% by weight. Occlusive agents are agents that increase moisture retention by providing a physical barrier to epidermal water loss, and should be present in any amount effective for this purpose. Exemplary occlusive agents include castor oil and other such oils, and a preferred range is 0.1-5% by weight, or 0.2-0.5% by weight. Ingredients with occlusive properties include petrolatum, waxes, oils and silicones. Emollients are ingredients that are added to improve the appearance of the skin by smoothing flaky skin cells. Exemplary emollients include, for example, olive oil esters. Emollients may be present in an amount ranging from 0.1-5% by weight.

Botanical extracts are added to provide a pleasant scent and possibly to provide a deodorizing or odor-masking property to the cleansing composition. The botanical extracts may comprise one or more of coffee seed extract, Punica Granatum extract, Croton Lechleri extract, Panax extract, and Crataegus Monogyna extract, a fragrance and a skin conditioning agent. For example, in some embodiments, a botanical extract may include coffee (Arabica) extract, and Punica Granatum extract. In another embodiment, the botanical extract may comprise Croton Lechleri extract. In yet another embodiment the botanical extract may comprise a combination of Panax extract and Crataegus Monogyna extract. Other cleansing combinations of these botanical extracts are possible, and other botanical extracts may be employed in addition to the foregoing. The botanical extracts are preferably present in an amount ranging from 0.01-5% by weight, or 0.05-2% by weight.

The cleansing composition may further include a preservative, such as sodium citrate or citric acid. These should be present in any amount effective to impart a preservative effect, such as a combined amount of about 0.5-5% % by weight. The composition may further include, for example, hydantoin, which may be present in an amount ranging from 0.1-0.4% by weight, or in any other suitable amount effective to impart a preservative effect. Phenoxyethanol, another preservative, may also present in any amount effective to provide a preservative effect, for example, an amount ranging from 0.1-1.0% by weight.

The cleansing composition should further include a fragrance, different from the botanical extract described hereinabove (although the fragrance itself may be a botanically-derived ingredient). Many suitable fragrances are known in the cosmetics and skin care industries and any suitable fragrance may be employed. The fragrance may be employed in any amount effective to provide a fragrant effect, such as an amount of about 1-2% by weight.

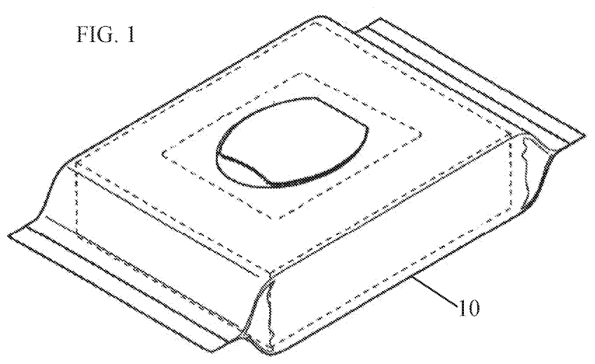

The cleansing cloths may be prepared for any suitable manner, typically, by dipping the base cloth material into a vat of the cleansing composition and allowing the cleansing composition to become impregnated into the cloth. The cloth may be in roll form when dipped, or may be already cut into individual cloths when dipped. Any suitable loading of the cleansing composition onto the cloths may be employed, for example, 12-17 grams of composition per cloth. In some embodiments the loading may be 15.6 g/cloth. Generally, otherwise, the method of manufacture of the cloths may be conventional. Upon the impregnation of the cleansing composition into the cloth, the cloths may be folded and packaged into polymeric resealable packages, using conventional packaging equipment. The cloths may be interlocked with one another or not interlocked with one another, depending upon preference. One such package is illustrated in FIG. 1.

The following non-limiting examples are provided to illustrate certain embodiments of the invention.

### EXAMPLES

### Example 1

Needle-punched 50% rayon/50% polyester cloth having a basis weight of 110 gsm was impregnated with a cleansing solution having the following formulation:

| Ingredient | % w/w |
|---|---|
| Water(Aqua) | 96.66 |
| Propylene Glycol | 1.0 |
| Parfum | 1.0 |
| PEG40 Hydrogenated Castor Oil | 0.2 |
| Punica Granatum Fruit Extract | 0.05 |
| Phenoxyethanol | 0.5 |
| DMDM Hydantoin | 0.22 |
| Olive oil PEG-7 esters | 0.2 |
| Coffee Arabica (Coffee) Seed Extract | 0.05 |
| Menthol | 0.02 |
| Sodium Citrate | 0.06 |
| Citric Acid | 0.04 |

Ten cloths impregnated with the above cleansing composition and having a size of roughly 8 in×8 in were packaged into a polymeric dispensing package.

### Example 2

Ten cloths were packaged in the same manner as in Example 1, except that the cleansing composition had the following formulation.

| Ingredient | % w/w |
|---|---|
| Water (Aqua) | 96.71 |
| Propylene Glycol | 1.0 |
| Parfum | 1.0 |
| PEG40 Hydrogenated Castor Oil | 0.2 |
| Croton Lechleri Resin Extract | 0.05 |
| Phenoxyethanol | 0.5 |
| DMDM Hydantoin | 0.22 |
| Olive oil PEG-7 esters | 0.2 |
| Menthol | 0.02 |
| Sodium Citrate | 0.06 |
| Citric Acid | 0.04 |

### Example 3

Ten cloths were packaged in the same manner as in Example 1, except that the cleansing composition had the following formulation.

| Ingredient | % w/w |
|---|---|
| Water(Aqua) | 96.66 |
| Propylene Glycol | 1.0 |
| Parfum | 1.0 |
| PEG40 Hydrogenated Castor Oil | 0.2 |
| Panax Quinquefolium Root | 0.05 |
| Extract | |
| Crataegus Mortogyna Fruit | 0.05 |
| Extract | |
| Phenoxyethanol | 0.5 |
| DMDM Hydantoin | 0.22 |
| Olive oil PEG-7 esters | 0.2 |
| Menthol | 0.02 |
| Sodium Citrate | 0.06 |
| Citric Acid | 0.04 |

It is thus seen that personal cleansing cloths may be provided in accordance with the foregoing teachings.

All methods described herein can be performed in any suitable order unless otherwise indicated herein or otherwise clearly contradicted by context. The use of any and all examples, or language describing an example (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

What is claimed is:

1. A package comprising a plurality of personal cleansing cloths, each cloth comprising a cloth material having a basis weight of at least 90 gsm and being impregnated with an aqueous cleansing composition, the aqueous cleansing composition comprising:
a skin cooling agent, the skin cooling agent being present in an amount effective to impart a cooling sensation;
a botanical extract comprising one or more of coffee seed extract, Punica Granatum extract, Croton Lechleri extract, Panax extract, and Crataegus Monogyna extract;
a fragrance; and
a skin conditioning agent.

2. The package of claim 1, wherein the skin cooling agent comprises one or more of menthol and menthyl lactate.

3. The package of claim 1, wherein the botanical extract comprises coffee seed extract and Punica Granatum extract.

4. The package of claim 1, wherein the botanical extract comprises Croton Lechleri extract.

5. The package of claim 1, wherein the botanical extract comprises Panax extract and Crataegus Monogyna extract.

6. The package of claim 1, wherein the skin conditioning agent comprises propylene glycol and one or more oils.

7. A personal cleansing method comprising:
providing a package comprising a plurality of personal cleansing cloths, each cloth comprising
a cloth material having a basis weight of at least 90 gsm and being impregnated with an aqueous cleansing composition, the aqueous cleansing composition comprising:
a skin cooling agent, the skin cooling agent being present in an amount effective to impart a cooling sensation;
a botanical extract comprising one or more of coffee seed extract, Punica Granatum extract, Croton Lechleri extract, Panax extract, and Crataegus Monogyna extract;
a fragrance; and
a skin conditioning agent;
removing a personal cleansing cloth from the package, and
self-cleansing using the personal cleansing cloth.

8. The method of claim 7, wherein the skin cooling agent comprises one or more of menthol and menthyl lactate.

9. The method of claim 7, wherein the botanical extract comprises coffee seed extract and Punica Granatum extract.

10. The method of claim 7, wherein the botanical extract comprises Croton Lechleri extract.

11. The method of claim 7, wherein the botanical extract comprises Panax extract and Crataegus Monogyna extract.

12. The method of claim 7, wherein the skin conditioning agent comprises propylene glycol and one or more oils.
